(19) United States
(12) Patent Application Publication
(10) Pub. No.: US 2018/0282811 A1
(43) Pub. Date: Oct. 4, 2018

(54) METHODS FOR DIAGNOSING, PROGNOSING, OR THERANOSING A CONDITION USING RARE CELLS

(71) Applicant: GPB Scientific, LLC, Richmond, VA (US)

(72) Inventors: Anne R. Kopf-Sill, Portola Valley, CA (US); Lena Wu, Los Altos Hills, CA (US); Roland Stoughton, San Diego, CA (US)

(73) Assignee: GPB Scientific, LLC, Richmond, VA (US)

(21) Appl. No.: 15/870,945

(22) Filed: Jan. 13, 2018

Related U.S. Application Data
(63) Continuation of application No. 15/204,693, filed on Jul. 7, 2016, now abandoned.
Application No. 15/204,693 is a continuation of application No. 14/633,522, filed on Feb. 27, 2015, now abandoned.
Application No. 14/633,522 is a continuation of application No. 13/803,741, filed on Mar. 14, 2013, now abandoned.
Application No. 13/803,741 is a continuation of application No. 12/595,949, filed on May 27, 2010, now abandoned.
(60) Provisional application No. 60/912,149, filed on Apr. 16, 2007.
(60) Provisional application No. 60/912,143, filed on Apr. 16, 2007.
(60) Provisional application No. 60/912,149, filed on Apr. 16, 2007.

(51) Int. Cl. C12Q 1/6883; G01N 33/574

(52) U.S. Cl. CPC C12Q 1/6883; G01N 2333/705; C12Q 2600/118; C12Q 2600/156; G01N 33/57492

(57) ABSTRACT

Described herein are methods for diagnosing, prognosing, or theranosing a condition using rare cells.

### CROSS-REFERENCE TO RELATED APPLICATIONS

This application claims the benefit of U.S. Provisional Application No. 60/912,147, filed Apr. 16, 2007, U.S. Provisional Application No. 60/912,143, filed Apr. 16, 2007, and U.S. Provisional Application No. 60/912,149, filed Apr. 16, 2007, which are hereby incorporated by reference.

### TECHNICAL FIELD

The invention is related to medical diagnostics and methods for diagnosing, prognosing, or theranosing a condition in a patient.

### BACKGROUND

Cancer is a disease marked by the uncontrolled proliferation of abnormal cells. In normal tissue, cells divide and organize within the tissue in response to signals from surrounding cells. Cancer cells do not respond in the same way to these signals, causing them to proliferate and, in many organs, form a tumor. As the growth of a tumor continues, genetic alterations may accumulate, manifesting as a more aggressive growth phenotype of the cancer cells. If left untreated, metastasis, the spread of cancer cells to distant areas of the body by way of the lymph system or bloodstream, may ensue. Metastasis results in the formation of secondary tumors at multiple sites, damaging healthy tissue. Most cancer death is caused by such secondary tumors.

Despite decades of advances in cancer diagnosis, prognosis and therapy, many cancers are not diagnosed, prognosed or treated properly. As one example, most early-stage lung cancers are asymptomatic and are not detected in time for curative treatment, resulting in an overall five-year survival rate for patients with lung cancer of less than 15%. However, in those instances in which lung cancer is detected and treated at an early stage, the prognosis is much more favorable. As another example, breast cancer is detected in a patient and then subjected to a therapeutic treatment using monoclonal antibodies. However, the patient doesn't respond to the therapeutic treatment.

Therefore, there exists a need to develop new methods for diagnosis, prognosis, and theranosis of cancer.

### INCORPORATION BY REFERENCE

All publications and patent applications mentioned in this specification are herein incorporated by reference to the same extent as if each individual publication or patent application was specifically and individually indicated to be incorporated by reference.

### SUMMARY OF THE INVENTION

In one aspect of the invention, a method for diagnosing, theranosing, or prognosing a condition in a patient comprises detecting a serum marker shed from a primary tumor in a first sample; enumerating one or more circulating tumor cells in a second sample from said patient; and diagnosing, prognosing, or therano sing the condition in said patient based on said detecting a serum marker and said enumerating one or more circulating tumor cells.

The first or second sample can be a blood sample. The first and second sample can be the same sample. The serum marker can be hTR, hTERT, TEP1, estrogen, epidermal growth factor, transforming growth factor, prostaglandin E2, estrogen-regulated proteins such as pS2, interleukins (eg., IL-10), S-100 protein, vimentin, epithelial membrane antigen, prostate specific antigen, bcl-2, CA15-3, CA 19-9, mucin core carbohydrate, Tn antigen, Tn-like antigen, alpha-lactalbumin, lipid-associated sialic acid, galactose-N-acetylgalactosamine, GCDFP-15, Le(y)-related carbohydrate antigen, CA 125, urokinase-type plasminogen activator, uPA related antigen, uPA related complex, uPA receptor, beta-glucuronidase, CD31, CD44 splice variants, blood group antigens, ABH, Lewis, MN, MK, DUPAN2, LCAP, TAG-12, TPA, TPS, carcinoembryonic antigen, squamous cell carcinoma antigen, tissue polypeptide specific antigen, sialyl TN mucin, placental alkaline phosphatase, BPC-1, or CC2.

Enumerating the number of CTCs in a sample from said patient can comprise flowing said sample through a microfluidic device that selectively enriches one or more circulating tumor cells. The microfluidic device can enrich one or more CTCs based on size, affinity, deformability, or shape. The method for diagnosing, theranosing, or prognosing a condition in a patient by detecting a serum marker shed can further comprise performing one or more nucleic acid analysis on said circulating tumor cells. The microfluidic device can comprise an array of obstacles and/or one or more binding moieties. The one or more binding moieties can comprise anti-EpCAM.

The method for diagnosing, theranosing, or prognosing a condition in a patient comprising detecting a serum marker can further comprise performing one or more nucleic acid analysis on said circulating tumor cells.

The method for diagnosing, theranosing, or prognosing a condition in a patient comprising detecting a serum marker can further comprise subjecting said patient to one or more therapeutic treatments; repeating said detecting a serum marker and said enumerating one or more circulating tumor cells; and diagnosing, prognosing or theranosing the condition in the patient.

In another aspect of the invention, a method for diagnosing, theranosing, or prognosing a condition in a patient comprises performing one or more nucleic acid analysis on a first sample obtained from said patient; enumerating one or more rare cells in a second sample from said patient; and diagnosing, theranosing, or prognosing the condition in said patient based on said enumerating one or more rare cells and said performing one or more nucleic acid analysis.

The first sample can be a biopsy sample, the second sample can be a blood sample, or the first and second sample can be the same sample. Performing one or more nucleic acid analysis can comprise SNP analysis, mRNA analysis, or sequencing. The one or more rare cells can comprise circulating tumor cells.

The one or more rare cells can be enriched using a microfluidic device. The microfluidic device can comprise one or more binding moieties and/or an array of obstacles. The one or more binding moieties can comprise anti-EpCAM.

The method for diagnosing, theranosing, or prognosing a condition in a patient comprising performing one or more nucleic acid analysis can further comprise subjecting said patient to one or more therapeutic treatments; repeating said performing one or more nucleic acid analysis and said enumerating one or more rare cells; and diagnosing, prognosing or theranosing the condition in the patient.

In one aspect of the invention, a method for diagnosing, theranosing, or prognosing a condition in a subject, comprises a) enriching one or more rare cells from a sample obtained from said subject using a microfluidic device; b) performing a first analysis of one or more cell subtypes of said one or more rare cells; and c) evaluating the result of said first analysis to make said diagnosis, theranosis, or prognosis.

The method for diagnosing, theranosing, or prognosing a condition in a subject comprising performing a first analysis of one or more cell subtypes can further comprise labeling one or more rare cells using a first label and labeling one or more cell subtypes using a second label.

The first label can be distinct from the second label. The first label and the second label can have a light absorption wavelength or a fluorescence emission wavelength that is separated by more than 5, 10, 25, 30, 40, or 50 nm. The first analysis can comprise enumerating the one or more cell subtypes. The cell subtypes can comprise circulating tumor cells, circulating tumor stem cells, circulating stem cells, or stem cells. The microfluidic device can comprise an array of obstacles and/or one or more binding moieties. The one or more binding moieties can comprise anti-EpCAM.

The method for diagnosing, theranosing, or prognosing a condition in a subject comprising performing a first analysis of one or more cell subtypes can further comprise subjecting said enriched one or more rare cells to one or more therapeutic treatments after step b), performing a second analysis of one or more cell subtypes, and evaluating the results of said first and second analysis to make said diagnosis, theranosis, or prognosis.

Steps a)-c) can be performed at a first time and a second time, and the results obtained from at the first time and the results obtained at the second time can be evaluated to make said diagnosis, theranosis, or prognosis.

The method for diagnosing, theranosing, or prognosing a condition in a subject comprising performing a first analysis of one or more cell subtypes can further comprise subjecting said patient to one or more therapeutic treatments between said first time and said second time.

In one aspect of the invention, a method for diagnosing, theranosing, or prognosing a condition in a patient comprises enriching one or more CTCs in a sample obtained from said patient; subjecting said one or more CTCs to one or more therapeutic treatments or culturing said one or more circulating tumor cells; and diagnosing, theranosing, or prognosing the condition in the patient.

The one or more CTCs can be enriched using a microfluidic device comprising an array of obstacles and/or one or more binding moieties. The one or more therapeutic treatments can comprise a chemotherapy agent. The one or more CTCs can be released or can be not released from the microfluidic device prior to culturing said one or more circulating tumor cells.

The method for diagnosing, theranosing, or prognosing a condition in a patient comprising subjecting said one or more CTCs to one or more therapeutic treatments or culturing said one or more circulating tumor cells can further comprise subjecting said one or more CTCs to one or more therapeutic treatments after said culturing said one or more circulating tumor cells; and/or identifying one or more therapeutic treatments based on the whether said CTCs respond to said one or more therapeutic treatments.

The method for diagnosing, theranosing, or prognosing a condition in a patient comprising subjecting said one or more CTCs to one or more therapeutic treatments or culturing said one or more circulating tumor cells can further comprise analyzing said one or more CTCs before and after said subjecting said one or more CTCs to one or more therapeutic treatments.

In another aspect of the invention, a business method comprises enriching one or more rare cells in a first sample obtained from a patient using a microfluidic device, wherein the microfluidic comprises an array of obstacles and/or one or more binding moieties; enumerating said one or more rare cells; analyzing a second sample from the patient by performing nucleic acid analysis or detecting a serum marker; diagnosing, theranosing, or prognosing a condition in the patient; and providing a report on said condition in exchange for a fee.

The invention provides for a kit for diagnosing, theranosing, or prognosing a condition in a patient comprising: microfluidic device comprising an array of obstacles and/or one or more binding moieties; and one or more reagents for performing nucleic acid analysis, detecting a serum marker, and/or culturing cells.

### BRIEF DESCRIPTION OF THE FIGURES

FIG. 1 shows a listing of markers.

FIG. 2 shows a listing of Sequence IDs.

### DETAILED DESCRIPTION OF THE INVENTION

### Sample and Sample Components

The present invention related to methods for diagnosing, prognosing, and staging conditions in a patient including cancer as selecting a therapy (theranosing) and monitoring treatment in patients. The methods herein utilize the fact that circulating rare cells, such as circulating tumor cells (CTCs), epithelial cells, and circulating stem cells, are an indicator and a source of various conditions in an organism. Thus the enumeration, characterization, and analysis of rare cells can be critical for diagnosing disease and disease states.

Rare cells can be obtained from a sample from a patient. A rare cell can be one that is up to 0.5%, 1%, 5%, or 10% of all cells in the sample. A sample can be any cellular, preferably, fluidic sample, from the patient. A typical sample is a blood sample. A fluidic sample from a patient or one that has been solubilized can be up to about 1, 2, 3, 4, 5, 6, 7, 8, 9, 10, 20, 50, 75, 100, 200, 500, 1000 or 1500 mL or greater than 5, 7.5, 10, 50, 75, 100, 500, or 750 mL.

Example of a rare cell include, but is not limited to, a circulating tumor cell (CTC), a circulating epithelial cell, a circulating stem cell, an undifferentiated stem cell, a cancer stem cell, a bone marrow cell, a progenitor cell, a foam cell, a mesenchymal cell, a circulating endothelial cell, a circulating endometrial cell, a trophoblast, a cancer cell, an immune system cell (host or graft), a connective tissue cell, a bacteria, a fungi, or a pathogen (e.g., bacterial or protozoa).

In one example, a rare cell is a circulating epithelial cell found in the blood stream of a patient. Such epithelial cell is exfoliated from a solid tumor can be found in very low concentrations in the circulation of a patient with cancer of the breast, colon, liver, ovary, prostate, and lung. Presence, quantity, and/or concentration of these cells in blood can be correlated with overall prognosis and/or response to therapy. Such an epithelial cell can be referred to as a circulating tumor cell. A CTC can be an early indicator of tumor expansion or metastasis before the appearance of a clinical symptom.

Enumeration and characterization of one or more rare cells, such as CTCs, using the devices and methods herein may be useful in assessing cancer diagnosis and prognosis including, early cancer detection, early detection of treatment failure, and detection of cancer relapse. Enumeration and characterization of one or more rare cells using the devices and methods herein may also be useful in selecting and monitoring therapy in a patient.

### Enrichment Devices

The methods herein contemplate taking a sample from a patient, such as a blood sample, and optionally enriching one or more rare cells from the sample using an enrichment device. An enrichment device (ED) is preferably a microfluidic device. Such device can selectively enrich rare cells from a sample based on one or more of their unique properties such as size, affinity, shape, and/or deformability.

In some instances, an enrichment device comprises an array of obstacles (e.g., obstacles arranged in two dimensions). The obstacles can be arranged uniformly or non-uniformly. The obstacles have microfluidic gaps between them. The gaps permit enrichment of rare cells based on size, affinity, shape, and/or deformability. For examples, obstacles may be configured to capture cells larger than a certain size (e.g., capture CTCs) based on differential hydrodynamic sizes of cells. (CTC's tend to be larger than the average blood cell.) Obstacles can be covered with one or more binding moieties that specifically bind cell surface markers of rare cells thereby selectively capturing them based on affinity. For example, an array of obstacles can have covered with anti-Ep-CAM antibodies that selectively bind epithelial cells, thereby enriching circulating epithelial cells from a blood sample. An enrichment device comprising an array of obstacles can preferably process up to 1, 2, 3, 4, 5, 6, 7, 8, 9, 10, 20, 50, 75, 100, 200, 500, 1000 or 1500 mL of a fluid sample within 5 hours, 4 hours, 3 hours, 2 hours, 1 hour, 30 minutes, or 10 minutes.

The microfluidic devices described herein can comprise an array of obstacles with an average gap between obstacles and a restricted gap between obstacles. The average gap length can be the average distance between adjacent obstacles. The restricted gap can have a distance between adjacent obstacles that is less than the average gap length. The number of restricted gaps can be up to 0.5%, 1%, 5%, 10%, 25%, or 50% of the total number of gaps between adjacent obstacles.

In some instances, the array comprises a plurality of subarrays that are situated in a staggered position with respect to one another to create a restricted gap and an expanded gap at a regular or irregular interval. The restricted gap can be used to slow down fast flowing cells.

In one embodiment, an array performs both size and affinity separation. Such array has obstacles or posts that become progressively closer to one another along the flow path. For example, the device can be a microfluidic device that comprises an array of obstacles that includes one or more subarrays of obstacles that are fluidly connected to one another in series. The subarrays of obstacles can be arranged such that a first subarray is positioned upstream of a second subarray, and the second subarray would be positioned upstream of a third subarray. The first subarray can comprise a first gap length between obstacles and the second subarray can comprise a second gap length between obstacles. The third subarray can have a third gap length between obstacles. The second gap length can be less than the first gap length. The third gap length can be less than the second gap length. Such an array can have multiple subarrays (e.g., at least 2, 3, 4, 5, 6, 7, 8, 9 or 10). The posts in such devices can be covered with one or more antibodies. In some instances, the array above is covered with anti-Ep-CAM antibodies, and optionally, anti-EGFR antibodies. The posts in such devices can be covered with two or more antibodies. The one, two or more antibodies can be in the same region (e.g., on the same obstacles) or in distinct regions (e.g., on different obstacles). When on different obstacles, the order of antibodies can be alternating along the flow path or perpendicular to the flow path.

The microfluidic device with an array of obstacles can be used to enrich one or more cells with a specified size range, for example, by retaining cells having a hydrodynamic size greater than 12, 14, 16, 18, or even 20 microns from a sample. Alternatively, a microfluidic device comprising an array of obstacles can enrich one or more cells having a hydrodynamic size greater than or equal to 6 microns and less than or equal to 12 microns.

The array of obstacles described above or one that does not separate cells by size can include one or more binding moieties on its surface to selectively bind the rare cells. A binding moiety can include a nucleic acid (e.g., DNA, RNA, PNA, or oligonucleotide), a ligand, a protein (e.g. a receptor, a peptide, an enzyme, an enzyme inhibitor, an enzyme substrate, an antibody, an immunoglobulin (particularly an antibody or fragment thereof), an antigen, a lectin, a modified protein, a modified peptide, a biogenic amine, a complex carbohydrate, or a synthetic molecule. Preferably, a binding moiety is an antibody that selectively binds a receptor of the rare cells of interest, e.g., epithelial cells or CTCs.

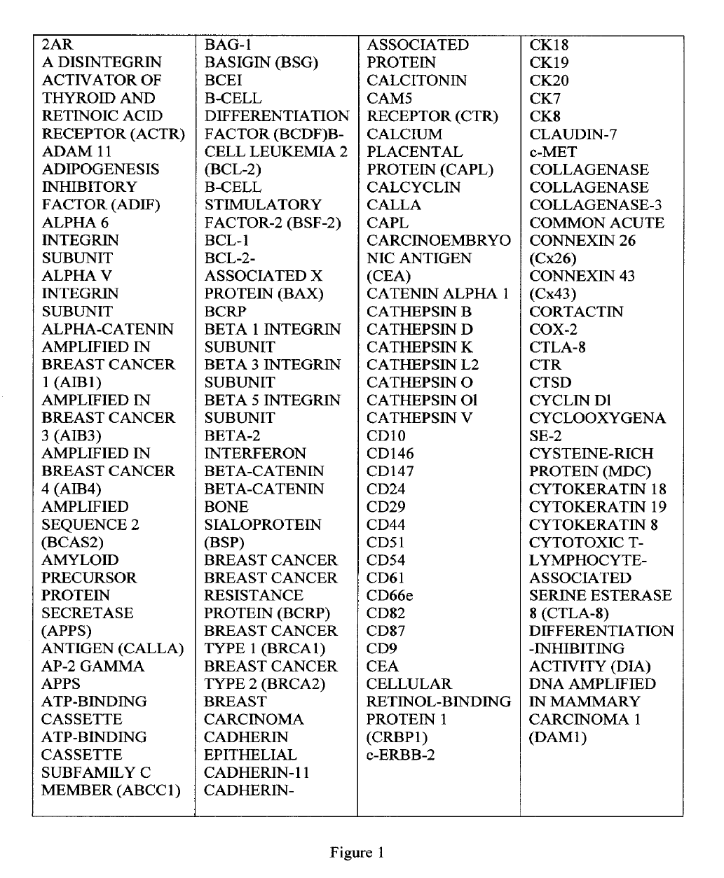

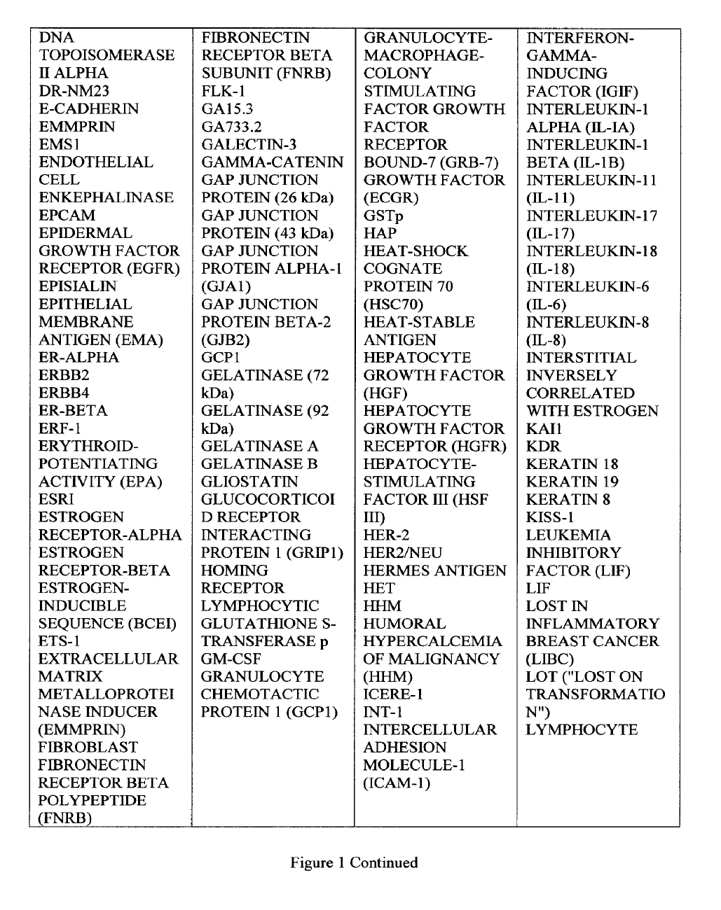

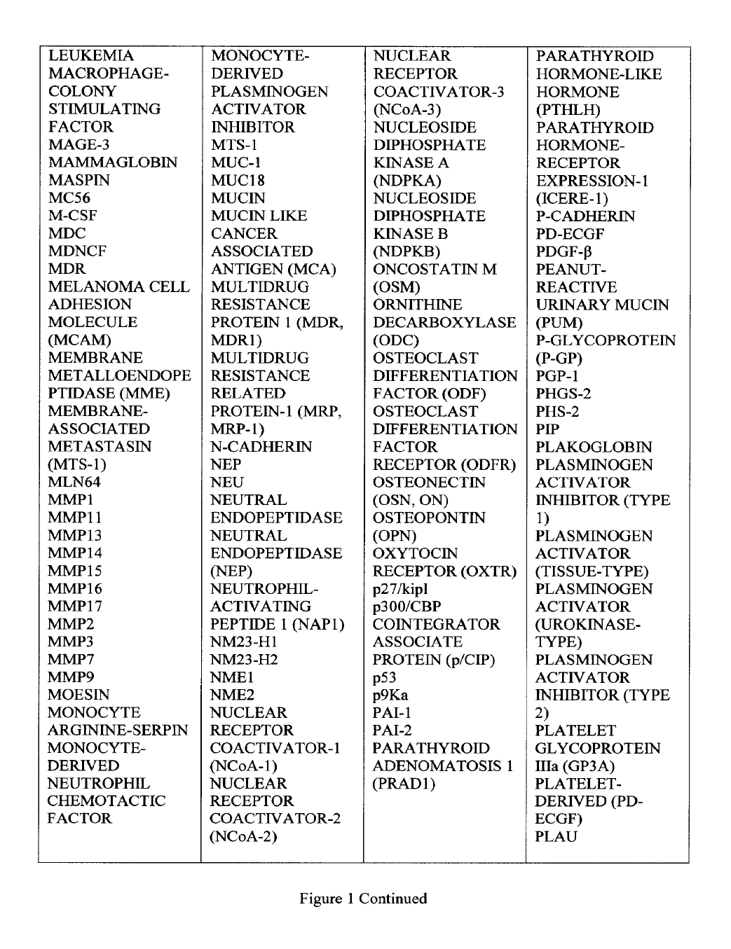

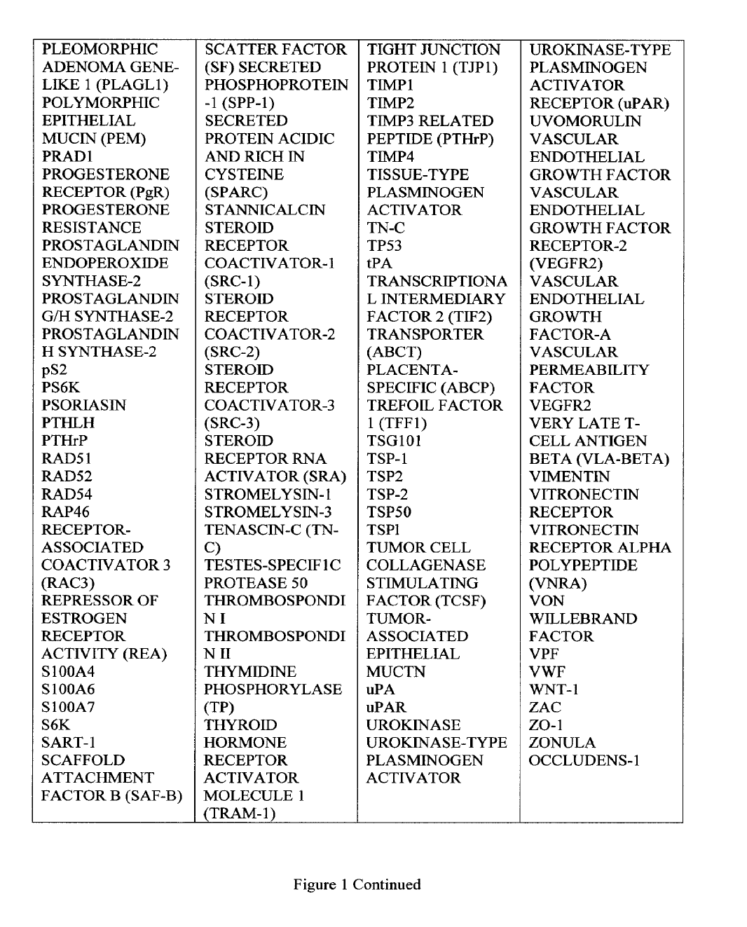

Examples of antibodies contemplated herein include, but are not limited to, anti-CD71, anti-CD235a, anti-CD36, anti-carbohydrates, anti-selectin, anti-CD45, anti-GPA, anti-antigen-i, anti-EpCAM, anti-E-cadherin, anti-Muc-1, or any antibody to a marker shown in FIG. 1. EpCAM may be referred to as the following: Ep-Cam, GA733-2, EGP, GP40, EPG2, KSA, 17-1A, C017-1A, Esa, TACSTD1, CD326, M4S1, MIC18, MK-1, TROP1, or hEGP-2.

Gentle handling of the sample by the microfluidic devices described herein can preserve the one or more enriched cells in a sample, prevent rupture of the one or more enriched cells, and/or prevent maturation or activation of the one or more enriched cells. The gentle handling can also permit allow for culturing of one or more enriched cells or downstream analysis of cellular material, including genetic material.

The microfluidic devices described herein can also include a lid or a port. The lid can be detachable, optically transparent, or optically opaque. The port can be used for delivering fluid to and removing fluid from a microfluidic device. The port can be removable.

Microfluidic devices and methods for enrichment of rare cells based on size, affinity, deformability, and shape are also described in co-pending US Application Publication No. 2006/051265 which is hereby incorporated by reference.

### Uses of Rare Cells

Rare cells enriched using one or more methods described herein or other methods known in the art can be used to diagnose or prognose a condition, theranose, or monitor treatment.

Diagnosing can comprise determining a condition of a patient. For example, a patient can be diagnosed with cancer or with another disease based on results from obtaining a sample from the patient, enriching a sample in one or more rare cells, and analyzing the one or more rare cells.

Prognosing can comprise determining the outcome of a patient's condition, the chance of recovery, or how the disease will progress. For example, a patient can obtain a prognosis of having a 50% chance of recovery based on results from obtaining a sample from the patient, enriching a sample in one or more rare cells, and analyzing the one or more rare cells.

Theranosis can comprise determining a therapy treatment for a condition. For example, a patient's therapy treatment can be chosen based on the response of one or more enriched cells that have been cultured and treated with a therapeutic agent.

The methods of the invention also comprise monitoring a patient over time for determining the recurrence of a condition in a patient. A sample can be obtained from a patient at various times, for example 1, 2, 3, 4, 5, 10, or 20 years after treatment and/or remission of a condition. The sample can be analyzed using methods and devices of the invention described herein. Recurrence of the condition can be determined by a change in an indicator. An indicator can be, for example, an increase in the number of rare cells enriched from the sample.

Any of the methods or fluidic devices described herein can be used for selecting a patient. Patients can be selected for inclusion or exclusion from clinical trials or for providing or not providing the patient a therapeutic treatment. A patient can be selected for a clinical trial or for treatment if, for example, the patient sample has more than a set number of rare cells. A set number can be an expected number based on healthy patients. A set number can also be an expected number of cells based on a sample from the same patient taken at a different time.

### Serum Marker Analysis

A method for diagnosing, theranosing or prognosing a condition in a patient comprises: (A) either (i) enumerating one or more rare cells in a sample from the patient, or (ii) performing a nucleic acid analysis on rare cells in a sample from the patient, and (B) detecting (quantitating) a serum marker in a blood sample from the patient.

The sample used for rare cell analysis can be derived from the same sample from the patient or from a different sample from the same patient as the one used for detecting (quantitating) a serum marker. Preferably both samples are blood samples, and optionally are derived from the same blood sample. For diagnosing a cancer condition, the rare cells are CTCs or epithelial cells.

Conditions can include, but are not limited to, hematological conditions, inflammatory conditions, ischemic conditions, neoplastic conditions, infections, traumas, endometriosis, and kidney failure (see, e.g., Takahashi et al., Nature Med. 5:434-438 (1999), Healy et al., Hum. Reprod. Update 4:736-740 (1998), and Gill et al., Circ. Res. 88:167-174 (2001)). Neoplastic conditions include, but are not limited to, prostate cancer, lung cancer, ovarian cancer, breast cancer, colorectal cancer, esophageal cancer, stomach cancer, small intestinal cancer, anal cancer, liver cancer, gallbladder cancer, pancreatic cancer, head and neck cancer, melanoma, uterine cervical cancer, uterine corpus cancer, vulva cancer, vaginal cancer, testicular cancer, penile cancer, urinary bladder cancer, kidney cancer, acute lymphoblastic leukemia, acute or chronic lymphocyctic or granulocytic tumor, acute myeloid leukemia, acute promyelocytic leukemia, adenocarcinoma, adenoma, adrenal cancer, basal cell carcinoma, bone cancer, brain cancer, bronchi cancer, cervical dysplasia, chronic myelogenous leukemia, epidermoid carcinoma, Ewing's sarcoma, gallbladder cancer, gallstone tumor, giant cell tumor, glioblastoma multiforma, hairy-cell tumor, hyperplasia, hyperplastic corneal nerve tumor, in situ carcinoma, intestinal ganglioneuroma, islet cell tumor, Kaposi's sarcoma, kidney cancer, larynx cancer, leiomyomater tumor, liver cancer, lymphomas, malignant carcinoid, malignant hypercalcemia, malignant melanomas, marfanoid habitus tumor, medullary carcinoma, metastatic skin carcinoma, mucosal neuromas, mycosis fungoide, myelodysplastic syndrome, myeloma, neural tissue cancer, neuroblastoma, osteogenic sarcoma, osteosarcoma, parathyroid cancer, pheochromocytoma, polycythemia vera, primary brain tumor, prostate cancer, rectum cancer, renal cell tumor, retinoblastoma, rhabdomyosarcoma, seminoma, skin cancer, small-cell lung tumor, soft tissue sarcoma, squamous cell carcinoma, stomach cancer, thyroid cancer, topical skin lesion, veticulum cell sarcoma, and Wilm's tumor.

The one or more rare cells (e.g., epithelial cells, CTCs, circulating tumor cells) can be enriched prior to enumeration or nucleic acid analysis using a microfluidic device. The microfluidic device can comprise an array of obstacles and/or one or more binding moieties, such as anti-EpCAM. The microfluidic device can enrich one or more CTCs based on size, affinity, deformability, and/or shape and may have any of the configurations described herein.

Steps (A) and (B) recited above can be repeated multiple times (e.g., before and after treatment, throughout a treatment regimen, etc.). A change in the amount of serum marker and a change in the number of rare cells or nucleic acid content (e.g., expression) of the rare cells can be used to diagnose, theranose, or prognose a condition in the patient.

When step (A) involves enumeration, such enumeration can take place using fluorescent probes specific to, e.g., nucleus, cytokeratin, CD-45. Enumeration can also be performed using any methods described herein. Enumeration of rare cell can be accomplished using any means known in the art or described herein. In some instances, rare cells are enriched using a microfluidic device prior to enumeration. Enumerating the number of CTCs in a blood sample from said patient can comprise flowing said sample through a microfluidic device that selectively binds said circulating tumor cells. The cells may be labeled and counted in the device or released from the device before labeling and counting.

Analysis techniques to perform the methods of analysis can include a variety of analytical techniques. A label can be used to detect a component of a cellular sample. The label can be a label conjugated to an antibody that targets any marker shown in FIG. 1. The label can target any protein, gene, or small molecule associated with a marker shown in FIG. 1. The label can bind to an analyte, be internalized, or be absorbed. Labels can include detectable labels. The detectable label can be detected based on electromagnetics, mechanical properties, electrical properties, shape, morphology, fluorescence, phosphorescence, magnetic properties, radioactive emission, etc. The label can include an antibody to a component of the sample and a fluorescent dye. The label can comprise an anti-cytokeratin antibody and phycoerythrin.

The number of rare cells in a sample, the change in number of rare samples over time or after therapy, and/or the genetic profile of rare cells can provide information about the course of a condition or can signal a change in a condition. This information can be used to generate a diagnosis, theranosis, or prognosis. In some cases, more than one type of cell (e.g., epithelial, endothelial, etc.) can be enumerated and a determination of a ratio of numbers of cells (e.g., endothelial and epithelial) or profile of various cells (CTC's, circulating tumor cells, and/or cells expressing a particular marker) can be obtained to generate a diagnosis, theranosis or prognosis.

When step (A) involves nucleic acid analysis, the analysis is performed on the one or more enriched rare cells can include RT-PCR, mRNA analysis, SNP analysis, or any other nucleic acid analyses described herein or known to those skilled in the art. For example, nucleic acid analysis can include RT-PCR to determine EGFR expression levels.

For step (B), examples of serum markers detected (quantitated) include, but are not limited to, CD26, hTR, hTERT, TEP1, estrogen, epidermal growth factor (EGF), transforming growth factor (TGF), prostaglandin E2 (PGE2), estrogen-regulated proteins such as pS2, interleukins (eg., IL-10), 5-100 protein, vimentin, epithelial membrane antigen, prostate specific antigen (PSA), bcl-2, CA15-3 (an aberrant form of polymorphic epithelial mucin (PEM)), CA 19-9, mucin core carbohydrates (eg., Tn antigen and Tn-like antigens), alpha-lactalbumin, lipid-associated sialic acid (LASA), galactose-N-acetylgalactosamine (Gal-GaINAC), GCDFP-15, Le(y)-related carbohydrate antigen, CA 125, urokinase-type plasminogen activator (uPA) and uPA related antigens and complexes (eg., LMW-uPA, HMW-uPA, uPA aminoterminal fragment (ATF), uPA receptor (uPAR) and complexes with inhibitors such as PAl-1 and PAl-2), beta-glucuronidase, CD31, CD44 splice variants, blood group antigens (eg., ABH, Lewis, and MN), MK (midkine), DUPAN2, LCAP, TAG-12, TPA, TPS, carcinoembryonic antigen (CEA), squamous cell carcinoma antigen (SCC), tissue polypeptide specific antigen (TPS), sialyl TN mucin (STN), placental alkaline phosphatase (PLAP), BPC-1, or CC2 (See, for example, U.S. Pat. Nos. 7,163,789; 7,128,877; 7,090,983; 7,078,188; 6,919,435; 6,770,445; 6,277,972 and 6,962,779 and Eskelinen et al, Anticancer Research, vol. 14, pp. 699-704, 1994; Sarandakou et al., 1997 Acta Oncol. 36:755; Sarandakou et al., 1998 Eur. J. Gynaecol. Oncol. 19:73; Meier et al., 1997 Anticanc. Res. 17(4B):2945; Kudoh et al., 1999 Gynecol. Obstet. Invest. 47:52; Ind et al., 1997 Br. J. Obstet. Gynaecol. 104:1024; Bell et al. 1998 Br. J. Obstet. Gynaecol. 105:1136; Cioffi et al., 1997 Tumori 83:594; Meier et al. 1997 Anticanc. Res. 17(4B):2949; Meier et al., 1997 Anticanc. Res. 17(4B):3019).

One skilled in the arts would be able to choose an appropriate serum marker for diagnosis, theranosis, or prognosis of a specified condition.

For example, breast cancer can be diagnosed, prognosed, or theranosed in a patient by enumerating CTCs (or epithelial cells) in a blood sample from a patient and measuring levels of one or more of the following serum markers in the same or a different blood sample from the patient: 260F9, 113F1, 266B2, 454C11, 33F8, 317G5, 520C9, or 260F-9-1C9. Similarly, breast cancer can be diagnosed, prognosed, or theranosed in a patient by analyzing gene expression in enriched epithelial cells in a blood sample from the patient and measuring levels of one or more of the above serum. One skilled in the art would know how to pick a serum marker from the list described above.

In one instance, lung cancer can be diagnosed, prognosed, or theranosed in a patient by enumerating CTCs (or epithelial cells) in a blood sample from a patient and measuring levels of one or more of the following serum markers in the same or a different blood sample from the patient: CYFRA 21-1, NSE, ProGRP, SCC, CEA, Tumor M2-PK, CRP, LDH, CA125, CgA, NCAM, or TPA. Similarly, lung cancer can be diagnosed, prognosed, or theranosed in a patient by analyzing gene expression in enriched epithelial cells in a blood sample from the patient and measuring levels of one or more of the above serum. One skilled in the art would know how to pick a serum marker from the list described above.

In another example, prostate cancer can be diagnosed, prognosed, or theranosed in a patient by enumerating CTCs (or epithelial cells) in a blood sample from a patient and measuring levels of one or more of the following serum markers in the same or a different blood sample from the patient: prostate specific membrane antigen (PSMA), KIAA 18, KIAA 96, prostate carcinoma tumor antigen-1 (PCTA-1), prostate-specific antigen (PSA), prostate secretory protein (PSP), prostate acid phosphatase (PAP), human glandular kallikrein 2 (HK-2), prostate stem cell antigen (PSCA), PTI-1, CLAR1 (U.S. Pat. No. 6,361,948), PG1, BPC-1, prostate-specific transglutaminase, cytokeratin 15, semenogelin II, NAALADase, PD-41, p53, TCSF (U.S. Pat. No. 5,856,112), p300, actin, EGFR, or HER-2/neu protein. Similarly, prostate cancer can be diagnosed, prognosed, or theranosed in a patient by analyzing gene expression in enriched epithelial cells in a blood sample from the patient and measuring levels of one or more of the above serum. One skilled in the art would know how to pick a serum marker from the list described above.

Ovarian cancer can be diagnosed, prognosed, or theranosed in a patient by enumerating CTCs (or epithelial cells) in a blood sample from a patient and measuring levels of one or more of the following serum markers in the same or a different blood sample from the patient: CA125, OVX1, inhibin, LASA-P, CA19-9, CEA, MB-70K, DM/70K, urinary gonadotropin factor, Ca130, PRL, or M-CSF. Similarly, ovarian cancer can be diagnosed, prognosed, or theranosed in a patient by analyzing gene expression in enriched epithelial cells in a blood sample from the patient and measuring levels of one or more of the above serum. One skilled in the art would know how to pick a serum marker from the list described above.

Colorectal cancer can be diagnosed, prognosed, or theranosed in a patient by enumerating CTCs (or epithelial cells) in a blood sample from a patient and measuring levels of one or more of the following serum markers in the same or a different blood sample from the patient: CRCA-1, CD44, CD45, CD44V3, CD44V6, and CD44V10 (U.S. Pat. No. 6,630,314), Carcinoembryonic Antigen (CEA); Alpha-Fetoprotein Modified for Increased Analytical Precision (AFP); Pancreatic Oncofetal Antigen (POA); Antigen Specific for #1116-N5′-19-9 Antibody; Lipid-Bound Sialic Acid (LSA); New oncogenes; Myc oncogenes; Ras oncogenes; Centocor CQA 72/4 (a measurement of tumor-associated Glycoprotein 72 (TAG-72) using epitope-specific antibody #B72-3), p53; Laminin-P1; Yale Col. Sr. Factor; Urinary Gonadotropin Peptide (UGP); ST receptor; CA19-9, CA 125, CK-BB, or Guanylyl Cyclase C. Similarly, ovarian cancer can be diagnosed, prognosed, or theranosed in a patient by analyzing gene expression in enriched epithelial cells in a blood sample from the patient and measuring levels of one or more of the above serum. One skilled in the art would know how to pick a serum marker from the list described above.

Oral cancer can be diagnosed, prognosed, or theranosed in a patient by enumerating CTCs (or epithelial cells) in a blood sample from a patient and measuring levels of one or more of the following serum markers in the same or a different blood sample from the patient: p53 responsive gene 2, β A inhibin, human α-1 collagen type I gene, placental protein 11, BENE protein, neuromedin U, flavin containing monooxygenase 2, runt-related transcription factor 1, α 2 collagen type I, fibrillin 1, absent in melanoma 1, non-voltage-gated 1 α sodium channel, protein tyrosine kinase 6, or epithelial membrane protein 1. Similarly, ovarian cancer can be diagnosed, prognosed, or theranosed in a patient by analyzing gene expression in enriched epithelial cells in a blood sample from the patient and measuring levels of one or more of the above serum. One skilled in the art would know how to pick a serum marker from the list described above.

### Nucleic Acid Analysis

A diagnosis, prognosis, or theranosis can be made based on nucleic acid analysis on a first sample obtained from a patient and enumeration of rare cells in a second sample obtained from the patient. The first sample can be a biopsy, a blood sample, or other sample. A biopsy can be from a primary tumor or secondary tumors. The second sample can be a blood sample, or the first and second sample can be the same sample (i.e., both a blood sample). The rare cells can be CTCs and be enriched using a microfluidic device. Nucleic acid analysis can be performed on the rare cells enriched using a microfluidic device.

The microfluidic device can comprise one or more binding moieties and/or an array of obstacles. The one or more binding moieties can comprise anti-EpCAM.

Enumeration can be performed using any methods as described herein.

Nucleic acid analysis performed on the first blood sample, e.g., a sample from a tumor, can include RT-PCR, single nucleotide polymorphism (SNP) analysis, mRNA analysis, sequencing, genome analysis, or any combination thereof. Nucleic acid analysis can also include analysis of chromosome copy number, somatic mutations, genetic abnormalities DNA methylation, microRNA levels, or any combination thereof. RT-PCR and mRNA analysis can be performed using any method known by those skilled in the arts. Nucleic acid analysis can include analysis of genetic abnormalities. Genetic abnormalities can be detected using a label that binds a nucleic acid such as, for example, a fluorescence label or a colorimetric label. Genetic abnormalities can be detected and/or analyzed using FISH, in situ hybridization, SNPs, PCR or mRNA microarrays or other methods known in the art. In one non-limiting example, the method further comprises detecting genetic abnormalities in rare cells. Detection of genetic abnormalities in cells can occur in said the microfluidic device.

The DNA polymorphism can be identified using a label to a unique tag sequence. In some cases, a nucleic acid tag comprises a molecular inversion probe (MIP). The methods for analyzing a nucleic acid can comprise performing one or more assays to analyze one or more nucleic acid molecules for a somatic mutation or a chromosome copy number change. A somatic mutation can include, for example, a deletion, an insertion or a point mutation. A chromosome copy number change can be an aneuploidy or a chromosome segmental aneuploidy.

The methods for analyzing a nucleic acid can comprise amplifying one or more regions of genomic DNA in a sample. In one such method, each of said one or more regions of genomic DNA can comprise one or more polymorphisms. Amplifying can be followed by, for example, ultra deep sequence analysis or quantitative genotyping (e.g., using one or more MIPs). Amplifying nucleic acids can be performed using any method known to those skilled in the arts.

Reagents for performing nucleic acid analysis can include nucleic acids and/or one or more primers. The primers can be used for amplifying one or more nucleic acid sequences or can be used as a probe to a complementary nucleic acid. Nucleic acids can be used as probes to complementary nucleic acids or be used as a template for other nucleic acid methods. The nucleic acids and primers can be single-stranded, double-stranded, or conjugated to one or more functional groups. The functional groups can be detectable labels or binding moieties. The nucleic acids can include any nucleic acid or marker described herein. The primers can include portions complementary to any nucleic acid or marker described herein.

Thus, in one example, diagnosing, prognosing, or theranosing a patient with breast cancer can be accomplished by performing a nucleic acid analysis on cells from a first sample obtained from the patient (e.g., breast tumor biopsy or other tissue biopsies) and enumerating the number of CTCs in a second sample obtained from the patient (e.g., blood sample). Nucleic acid analysis performed on the first sample can be associated with one or more nucleic acids including, but not limited to, a gene encoding ERBB2, SED. ID. NOs. 70-97 of Patent Application Publication US 2003/0190656, SED. ID. NOs. 1-56 of Patent Application Publication US 2004/0214179, or SED. ID. NOs. 112-198 of Patent Application Publication US 2007/0031873. It should be noted that the first biopsy can be from a biopsy outside the breast region, but any of the above nucleic acid regions can be analyzed to determine origin of the cancer.

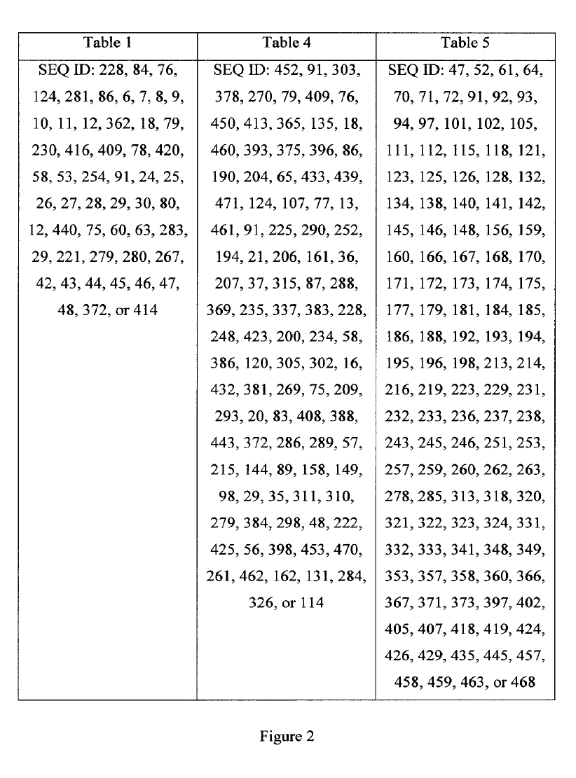

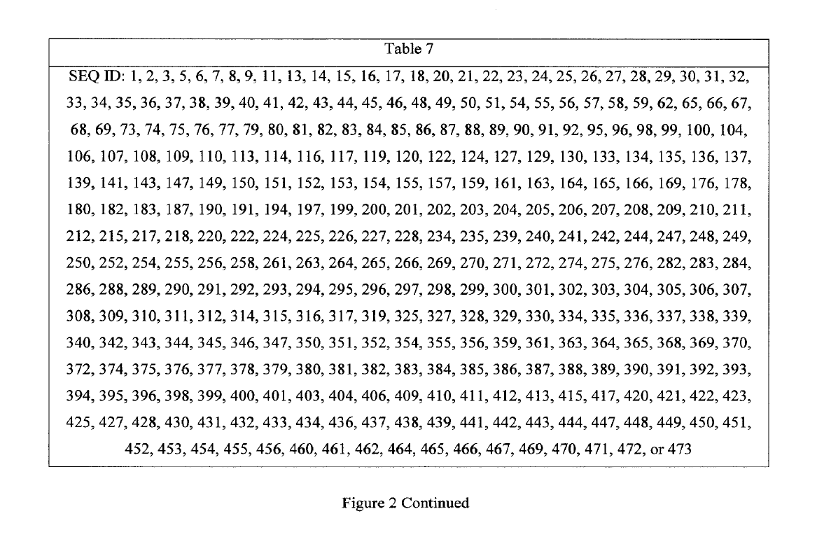

In another example, a patient can be diagnosed or prognosed with lung cancer or a theranosis can be made by performing a nucleic acid analysis on cells from a first sample obtained from the patient (e.g., lung tumor biopsy) and enumerating the number of CTCs in a second sample obtained from the patient (e.g., blood sample). Nucleic acid analysis performed on the first sample can be associated with, e.g., sequences shown in FIG. 2, which are sequencing listings from Table 1, Table 4, Table 5, and Table 7 of U.S. Patent Application Publication No. 2006/0252057.

In another example, a patient can be diagnosed or prognosed with ovarian cancer or a theranosis can be made by performing a nucleic acid analysis on cells from a first sample obtained from the patient (e.g., ovarian tumor biopsy) and enumerating the number of CTCs in a second sample obtained from the patient (e.g., blood sample). Nucleic acid analysis performed on the first sample can be associated with, e.g., sequences associated with BRCA1, BRCA2, CD72 (SEQ ID NO: 805), SLC25A11 (SEQ ID NO: 544), LCN2 (SEQ ID NO: 545-547), PSTP1P1(SEQ ID NO: 538-540), SIAHBP1 (SEQ ID NO: 543), UBE1 (SEQ ID NO: 533), WAS (SEQ ID NO: 524-526), IDH2 (SEQ ID NO: 541-542), PCTK1 (SEQ ID NO: 527-528), or SEQ ID NOs: 18-19, 30-31, 50-51, 52-54, 55-57, 58-59, 60, 68-69, 74-76, 85-86, 87-88, 89-91, 92-93, 94-95, 97-99, 122-123, 133-135, 149-151, 164-166, 167-168, 169-170, 174-175, 176-178, 179-180, 181-182, 190-192, or 199-201 of Patent Application Publication US 2005/0095592. One skilled in the art would know how to pick a nucleic acid from the list described above.

In another example, a patient can be diagnosed or prognosed with prostate cancer or a theranosis can be made by performing a nucleic acid analysis on cells from a first sample obtained from the patient (e.g., prostate tumor biopsy) and enumerating the number of CTCs in a second sample obtained from the patient (e.g., blood sample). Nucleic acid analysis performed on the first sample can be associated with, e.g., D1S235, D1S2678, D1S2785, D1S321, D1S2842 of chromosome 1, D1S252, D1S498, D1S305, D1S484, D1S196 of chromosome 1, D2S155, D2S325, D2S2242, D2S2321, D2S317, D2S2319, D2S2382, D2S2249, D2S163, D2S339 of chromosome 2, D4S405, D4S2974, D4S2996, D4S428, D4S2978, D4S3019, D4S1592, D4S398, D4S2987, D4S3004, D4S3018, D4S392, D4S1543 of chromosome 4, D5S2002, D5S2117, D5S393, D5S414, D5S2011, D5S2017, D5S436, D5S2090, D5S2013 of chromosome 5, D11S898, D11S927,D11S908, D11S1345, D11S934, D11S1320 of chromosome 11, D13S1290, D13S1283, D13S1230, D13S1234, D13S265, D13S1300, D13S281 of chromosome 13, or all polymorphic markers localized in the regions situated between the above markers. One skilled in the art would know how to pick a nucleic acid from the list described above.

In another example, a patient can be diagnosed or prognosed with colorectal cancer or a theranosis can be made by performing a nucleic acid analysis on cells from a first sample obtained from the patient (e.g., colorectal tumor biopsy) and enumerating the number of CTCs in a second sample obtained from the patient (e.g., blood sample). Nucleic acid analysis performed on the first sample can be associated with, e.g., SED. ID. NOs. 1-33, 35-36, and 38-41 of Patent Application Publication US 2003/0186303, SEQ ID NOs. 42-49 of Patent Application Publication US 2003/0186302, SEQ ID NOs. 1-4 of Patent Application Publication US 2004/0191782, or SEQ ID NOs. 7-13 of Patent Application Publication US 2005/0048494. One skilled in the art would know how to pick a nucleic acid from the list described above.

Other conditions can be associated with one or more of SED. ID. NOs. 1-30, 32, 34, and 98 of US Patent Application Publication No. 2003/0194733, SED. ID. NOs. 1-5, 10-13, 16-17, 19-23, 45-46, 83, and 85 of U.S. Pat. No. 6,218,529, SED ID. NOs. 1 and 3 of U.S. Pat. No. 5,783,403, or SED ID. NOs. 3 and 4 of U.S. Pat. No. 5,882,876, each of which sequences are hereby incorporated by reference. One skilled in the art would know how to pick a nucleic acid from the list described above.

### Analyzing Cell Subtypes

A method for diagnosing, theranosing, or prognosing a condition in a subject can comprise obtaining a sample from the subject, enriching rare cells from the sample, and analyzing or further enriching a subtype of the rare cells for purposes of making the diagnosis or prognosis or theranosis.

For example, diagnosis or prognosis of a cancer in a patient can be determined by enriching a set of rare cells using a microfluidic device, e.g., one that comprises an array of obstacles such that cells having a larger hydrodynamic size than most blood cells are captured based on size or one that comprises an array of obstacles covered with one or more binding moieties that selectively bind the rare cells based on their unique cell surface markers. In some instances, the microfluidic device comprises an array of obstacles covered with anti-Ep-CAM antibodies and the rare cells enriched are epithelial cells.

The enriched cells are then analyzed to detect one or more subtypes of rare cells. A rare cell subtype can include any type of cell classification based on a phenotype, a genotype of the cell, or any combination thereof, including, but not limited to, circulating cancer stem cells, circulating cancer non-stem cells, tumorigenic cells, non-tumorigenic cells, apoptotic cells, non-apoptotic cells, terminal cells, non-terminal cells, proliferative cells, non-proliferative cells, cells derived from specific tissues, cells derived from specific cancer tissues, disseminated cancer cells, micrometastasized cancer cells, or cells associated with a condition. Other examples of subtypes of rare cells include those of specific tissue of origin such as circulating endothelial cells or circulating lung, liver, breast or prostate cancer cells. Other cell classifications and cell subtypes can include cells with specific cancer phenotypes. For example, breast cancer cells are known to have at least 6 different phenotypes, such as luminal/epithelial, basal/myoepithelial, mesenchymal, ErbB2, hormonal, and hereditary. Phenotypes of a cancer cell are discussed in Patent Application Publication US 2004/0191783.

Rare cell subtypes can be detected or analyzed using any means known in the art, including pathological analysis, or via one or more labels specific to a subtype marker. Useful subtype markers include, but are not limited to c-kit, KIT, SPARC, SPARC, PDGFR, PDGFRA, PR, HSPCA, HIF1A, TOP2B, TOP1, TOP2A, VDR, GART, NFKBIA, SRC, NFKB1, TYMS, MGMT, ADA, RRM2, Her2/Neu, ER, PR, EGFR, Androgen Receptor, CD52, CD25, P-glycoprotein, ZAP70, CDW52, LCK, AR, DMNT3B, RRM2, DCK, FYN, RXRB, HDAC1, RAF1, EPHA2, ERCC1, MGMT, CD33, IL2RA, TK1, TYMS, NFKB1, ER003, YES1, ERBB2, FOLR2, ESR1, VEGF, ABCG2, TNF, OGFR, VHL, DNMT1, SSTR1, SSTR5, PDGFRB, SSTR4, DHFR, RXRG, SSTR2, NFKB2, DNMT3A, ABCC1, BCL2, SSTR3, VEGF, ECGF1, PDGFC, POLA, CES2, MS4A1, KDR, CDA, GSTP1, SSTR4, MLH1, RARA, PTGS2, PGR, ASNS, NFKBIA, RRM1, PTEN, FLTI, MSH2, VDR, BRCA1, TOP2A, TXNRD1, BRCA2, RRM2B, LYN, HF1A, HSPCA, BCL2 or a combination thereof. One skilled in the art would know how to pick a marker from the list described above.

Other subtype markers that can be used include H-ras, K-ras, N-ras, c-myc, bcr-abl, fms, src, fos, sis, jun, erb-B-1, VHL, PML/RAR, AML1-ETO, EWS/FLI-1, EWS/ZRG, p53, RB, MCC, APC, DCC, NF1, WT, alpha-feto protein (AFP), carcinoembryonic antigen (CEA), TAG-72, CA 19-9, CA-125, prostate specific antigen (PSA), prostate specific membrane antigen (PSMA), CD44, hcg (human chorionic gonadotropin), MAGE 1, MAGE 2, MAGE 3, MAGE 4, GP-100, MAGE 6, NUC 18, P97, tyrosinase mRNA, keratin 19 mRNA, telomerase RNA, RNA associated with heterogenous nuclear ribonucleoprotein Al (hn RNP-A1) and A2/B1 (hn RNP-A2/B1) complexes, heterogenous nuclear ribonucleoprotein K (hn RNP-K), c-myc oncogene RNA, B38.1, annexin V, Notch 4, CD9, CD24, MUC 1, CD49F, CD62P, P-glycoprotein, Notch 1, 520C9, 260F9 and 317G5. One skilled in the art would know how to pick a marker from the list described above.

In one example, a subtype of disseminated cancer cells or micrometastasized cancer cells are detected from a population of enriched rare cells by detecting a marker such as CEA, CK20, MUC1, tyrosinases, MAGE3, bFGF, bFGF-R, VEGF, VEGF-R1, VEGF-R2, MMP2, TIMP3, p53, erb-B2, c-myc, K-ras, RB, APC or DCC. One skilled in the art would know how to pick a marker from the list described above.

A subtype of cancer stem cells can be distinguished from cancer cells that are non-stem cells using the following criteria. They express (a) express CD44; (b) do not express detectable levels of one or more LINEAGE markers selected from among CD2, CD3, CD10, CD14, CD16, CD31, CD45, CD64, and CD140b; and (c) do not express CD24 or express low levels of CD24 (see, e.g., U.S. Pat. No. 6,984,522).

Other examples of rare cell subtypes include those that express a marker such as Ber-Ep4, CD34+, EpCAM, E-Cadherin, Mucin-1, Cytokeratin 8, EGFR, Leukocyte associated receptor (LAR), CD105, CD106, CD144, CD146, TEM1, TEM5, TEM8, CD133, GA733-2, Claudin-7, cytokeratin, p27, Ki67, VEGF, epidermal growth factor, epithelial membrane antigen, estradiol, estrogen, progesterone, androgen, members of tumor necrosis factor superfamily, ferritin, follicle stimulating hormone, actin, gastrin, heat shock proteins, lactoferrin, lamin B1, lutenizing hormone, tyrosine kinases, MAP kinase, microtuble associated proteins, c-Myc, myelin basic protein, myoglobulin, p16, cyclin-dependent kinases, p21, p53, proliferation-associated nuclear antigen, pancreatic polypeptides, proliferating cell nuclear antigen, prostatic acid phosphastase, prostate specific antigen, pS2, reinoblastoma gene product, S-100 protein, small cell lung cancer antigen, serotonin, somatostatin, oncogenes, tumor-associated probes, alpha fetal protein, P2 microglobulin, CA 19-9 antigen, CA 125 antigen, CA 15-3 antigen, CEA, Cathepsin D, p300 tumor-related antigen, collagen, melanoma, HIVIB45, HER-2/neu, p185, apoptotic genes and/or proteins, members of Bc1-2 subfamily, members of Bax subfamily, members of Bh3 subfamily, mitochondrial DNA, a telomerase, a nuclear matrix protein, or a microRNA which the remaining rare cells do not (or not at the same level). One skilled in the art would know how to pick a marker from the list described above.

Analysis of a rare cell subtype can comprise enumeration, nucleic acid analysis, protein composition analysis, etc. Enumeration can be performed using a detectable label that selectively binds to the rare cell subtype. The labeled cells are then detected and counted using any means known in the art. A nucleic acid analysis of a rare cell subtype can include performing gene expression analysis, SNPs analysis, and ultra deep sequencing analysis on such cells.

In some instances, the enumeration of rare cell subtype(s) by itself can be used as a diagnosis or prognosis of cancer.

In some instances, the enumeration of the rare cell subtype(s) at two different points in time can be used to monitor treatment. For example, if the number of circulating cancer stem cell (a subtype of CTCs) increases between a first sample collected before therapy or at the beginning of treatment and a second sample collected at a later point in time (e.g., after treatment), it can be concluded that the treatment is not helpful. Similarly, a baseline of circulating cancer stem cells in determined at the end of a treatment regimen and a subsequent sample obtained has an increase number of circulating cancer stem cells; there is an indication of cancer relapse.

Rare cell subtypes, such as circulating cancer stem cells, can also be isolated using any means known in the art or described herein (e.g., by flowing a sample through an array of obstacles covered with binding moieties that selectively bind the rare cell subtype, e.g., anti-CD44). Enriched or isolated rare cell subtypes can be used for therapy selection or to monitor treatment by enriching rare cells from a sample from a patient, subjecting one or more rare-cell subtypes from the rare cells enriched to therapeutic agent(s), observing the effects, and determining therapy based on the effect observed. hi some instances, the above is repeated over a course of a therapy to continuously monitor the efficacy of a treatment. (Cancer cells may mutate during a course of treatment and the number of cells in a subtype could increase or the nucleic acid composition of a subtype could change, indicating a need to change treatment.)

In some instances, enumeration of rare cell subtypes is combined with one or more other methods described herein, such as measuring a serum marker or performing a nucleic acid analysis on a tumor biopsy. (See discussion above)

In some instances, nucleic acid analysis can be performed on the enriched or isolated rare cell subtypes. Results from such nucleic acid analysis can be combined with enumeration of rare cell subtypes to diagnose, prognose or theranose.

As described above, rare cells can be enriched using a microfluidic device, including any of those described herein. An analysis of a cell subtype that is a portion of one or more rare cells enriched from a sample obtained from a patient can be repeated over time for diagnosis, prognosis, or theranosis of a condition in a patient.

### Selection of a Therapeutic Treatment and Prognosis

In some instances, the present invention contemplates selecting a therapeutic treatment and optionally prognosing a condition by enriching one or more rare cells (e.g., CTCs) from a patient sample (e.g., blood sample), subjecting the rare cells to one or more therapeutic treatments; and determining a treatment course based on results from the above.

When cells are enriched in a microfluidic device, e.g., by selective capture in an array of obstacles using size and/or affinity using any device described herein, one may subject them to therapeutic treatment(s) while they are still within the device or after they are released from the device. Moreover, rare cells enriched in a microfluidic device may be first cultured prior to being subjected to therapeutic treatment. The one or more rare cells can be analyzed before and after being subjected to one or more therapeutic treatments.

For example, enriched rare cells may be subject to analysis subsequently, they may be subject to one or more therapeutic agents, and subsequently, additional analysis may be performed on the cells to detect a change in genetic profile. Results from the first and second analysis can be used for diagnosis, theranosis, or prognosis of a patient condition.

Analysis methods contemplated herein include nucleic acid analysis (e.g., gene expression analysis), protein analysis, lipid analysis, cell enumeration, cell morphology, pleomorphism, somatic mutation, cell adhesion, cell migration, binding, division, protein phosphorylation, protein glycosylation, mitochondrial abnormalities, cell profiling, genetic profiling, telomerase activity, levels of a nuclear matrix protein or any analysis method described herein.

The therapeutic agents applied to the rare cells include, but are not limited to, chemotherapy agents or radiation as well as other conditions such as heat, radio waves, etc.

Examples of chemotherapy agents include, but are not limited to, doxcetaxel, platinum-based chemotherapy such as platin, carboplatin, ifosfamide, satraplatin and oxaliplatin, taxane, estramustin, doxorubicin, gemcitabine, Rubitecan, anthracycline- and taxane-based polychemotherapies or target-specific trastuzumab with or without endocrine manipulation with or without PMRT, virorelbine, 5-fluorouracil, levamisole, leucovorin or semustine (methyl CCNU). One skilled in the art would know how to pick a chemotherapy agent from the list described above.

Examples of radiation include, but are not limited to, external beam or braquitherapy, thoracic radiotherapy, radiation therapy with charged particles, interstitial brachytherapy, Mammosite device, 3-dimensional conformal external radiation and intraoperative radiotherapy. After therapeutic agents are applied to the rare cells, the rare cells are analyzed to determine efficacy of the treatment. Treatment selection may be based on identifying one or more therapeutic agents that preferentially kill at least 10%, 20%, 50% or 90% of all rare cells enriched. The therapeutic treatment can be a therapeutic treatment targeted to a type of cancer described herein.

The cancer can be prostate cancer and the one or more therapeutic treatments can be heat shock protein 90 (HSP90) inhibitors, chemotherapy (e.g., doxcetaxel, platinum-based chemotherapy such as platin, carboplatin, satraplatin and oxaliplatin, taxane, estramustin), prednisone or prednisolone, cholesterol-lowering drugs such as statins, leutinizing hormone-releasing hormone (LHRH) agonists, RNAi therapy, whole tumor cells genetically modified to secrete granulocyte macrophage—colony stimulating factor (GM-CSF) (also known as GVAX) or a combination thereof. One skilled in the art would know how to pick a therapeutic treatment from the list described above.

The cancer can be ovarian cancer and the one or more therapeutic treatments can be chemotherapy (e.g., doxorubicin, gemcitabine, Rubitecan, and platinum-based chemotherapeutics such as cisplatin, carboplatin and oxaliplatin), melphalan, paclitaxel, topoisomerase I inhibitors such as topotecan and irinotecan, taxane-based therapy, hormones, radiation therapy, whole body hypothermia, isoflavone derivatives such as Phenoxodial, cytotoxic macrolides such as Epothilones, angiogenesis inhibitors such as bevacizumab, signal transduction inhibitors such as trastuzumab, gene therapy, RNAi therapy, immunotherapy, monoclonal antibodies, phosphatidylinositol-like kinase inhibitors such as rapamycin or a combination thereof. One skilled in the art would know how to pick a therapeutic treatment from the list described above.

The cancer can be lung cancer and the one or more therapeutic treatments can be radiotherapy (e.g., thoracic radiotherapy, radiation therapy with charged particles, Uracil-tegafur and Platinum-based chemotherapy (e.g., cisplatin, carboplatin, oxaliplatin, etc.) and vinorebline, Erlotinib (Tarceva), Gefitinib (Iressa), anti-epidermal growth factor receptor antibodies (e.g., Cetuximab), anti-vascular endothelial growth factor antibodies (e.g., Bevacizumab), small molecule inhibitors of tyrosine kinases, direct inhibitors of proteins involved in lung cancer cell proliferation, Aurora kinase inhibitors, laser-induced thermotherapy, RNAi therapy, whole tumor cells genetically modified to secrete granulocyte macrophage—colony stimulating factor (GM-CSF) (also known as GVAX) or a combination thereof. One skilled in the art would know how to pick a therapeutic treatment from the list described above.

The cancer can be breast cancer and the one or more therapeutic treatments can be monoclonal antibodies (e.g., Her-2 antibodies, herceptin), hypoxic cells, adjuvant chemotherapy such as single agent chemotherapy or combination chemotherapy (e.g., anthracycline- and taxane-based polychemotherapies or target-specific trastuzumab with or without endocrine manipulation with or without PMRT, virorelbine), selective estrogen receptor modulators such as Tamoxifen and Raloxifene, allosteric estrogen receptor modulators such as Trilostane, radiation (e.g., interstitial brachytherapy, Mammosite device, 3-dimensional conformal external radiation and intraoperative radiotherapy), Aromatase inhibitors that suppress total body synthesis (e.g., anastrozole, exemestane and letrozole), RNAi therapy, intravenous analogs of rapamycin that are immunosuppressive and anti-proliferative such as Temsirolimus (CCI779) or a combination thereof. One skilled in the art would know how to pick a therapeutic treatment from the list described above.

The cancer can be colon cancer and the one or more therapeutic treatments can be radiation therapy, and chemotherapy (e.g., 5-fluorouracil, levamisole, leucovorin or semustine (methyl CCNU)), N-[2-(dimethylamino)ethyl]acridine-4-carboxamide and other related carboxamide anticancer drugs; non-topoisomerase II inhibitors, liposomal topotecan, taxane class of anticancer agents (e.g., paclitaxel or docetaxel), a compound of the xanthenone acetic acid class (e.g., 5,6-dimethylanthenone-4-acetic acid PMAA), laminarin, site-selective cyclic AMP Analogs (e.g., 8-chloroadenosine 3′,5′-cyclic phosphate), pyranoindole inhibitors of Cox-2, carbazole inhibitors of Cox-2, tetrahydrocarbazole inhibitors of Cox-2, indene inhibitors of Cox-2, localized inhibitors of NSAIDS (e.g., anthranilic acids, aspirin (5-acetylsalicylic acid), azodisal sodium, carboheterocyclic acids, carprofen, chlorambucil, diclophenac, fenbufen, fenclofenac, fenoprofen, flufenamic acid, flurbiprofen, fluprofen, furosemide, gold sodium thiomalate, ibuprofen, indomethacin, indoprofen, ketoprofen, lonazolac, loxoprofen, meclofenamic acid, mefanamic acid, melphalan, naproxen, penicillamin, phenylacetic acids, proprionic acids, salicylic acids, salazosulfapyridine, sulindac, tolmetin, a pyrazolone butazone propazone NSAID, meloxicam, oxicams, piroxicam, feldene, piroxicam beta cyclodextran, tenoxicam. etodolac, and oxaprozin), an inhibitor of HER-2/neu, RNAi therapy, GM-CSF, monoclonal antibodies (e.g., anti-Her-2/neu antibodies, anti-CEA antibodies, A33 (HB 8779), 100-210 (FIB 11764) and 100-310 (HB 11028)), hormonal therapy, pyrimidineamines, camptothecin derivatives (e.g., CPT-11), folinic acid (FA), Gemcitabine, Ara-C, platinum-based chemotherapeutics such as cisplatin, carboplatin and oxaliplatin, a cGMP-specific phosphodiesterase inhibitor. One skilled in the art would know how to pick a therapeutic treatment from the list described above.

The one or more rare cells can be cultured prior to being subjected to one or more therapeutic treatments. Culturing the one or more cells and, thus expanding the population can provide a larger number of cells to be analyzed. Cultured cells can be split into one or more sample in order to analyze response or sensitivity to one or more therapeutic treatments.

The methods contemplated herein comprise enriching one or more rare cells using any of the microfluidic devices as described herein. The enriched cells can then be cultured on the microfluidic device or released from the device and cultured in a separate vessel. The cultured cells are then subjected to any of the therapeutic agents described above. When cells are cultured in the microfluidic device, one or more ports can be plugged and a cell culture medium can be flowed into the device for culturing the one or more cells without first removing the one or more cells from the microfluidic device.

Alternatively, a lid can be removed from the microfluidic device, if present, and the microfluidic device may be placed in a culturing dish for culturing the one or more cells. Preferably, the device is flooded with a moiety (e.g., an antigen such as EpCAM or any other shown in FIG. 1) to bind any unbound affinity agents (e.g., antibodies), one or more outlet ports can be plugged and the one or more cells can be cultured as described above. As the cells retained on the device divide, daughter cells will be sloughed off.

The cells can be cultured using appropriate conditions. Media, temperature and carbon dioxide conditions are well known for cancer cells and would be utilized for culturing the one or more cells (e.g., U.S. Pat. Nos. 7,132,288; 6,777,230; and 5,023,172). Briefly, cells can be cultured in RPMI 1640 with 2 mmol/L L-glutamine, supplemented with 10% fetal bovine serum, 1 mmol/L sodium pyruvate, 100 units/mL penicillin, 100 μg/ml Fungizone. Cells can be incubated at 37° C. with 5% CO2 and maintained in log phase growth.

Therapeutic agents are administered to a patient based on results from the assays performed above on the cultured enriched rare cells.

For example, a blood sample can be obtained from a patient and then contacted with a microfluidic device comprising an array of obstacles and one or more binding moieties including anti-EpCAM and/or anti-EGFR. One or more rare cells can be retained or enriched by the microfluidic device and then analyzed using any of the analysis methods described herein. The analysis methods can include enumeration of the one or more rare cells and nucleic acid analysis of the one or more rare cells. Nucleic acid analysis, or any other analysis method, can be used to diagnose, prognose, or theranose a condition of the patient. An excess of binding antigens, such as EpCAM and/or EGFR are then flowed through the microfluidic device and bind to the one or more binding moieties of the microfluidic device. One or more culturing agents can be added to the microfluidic device for culturing the one or more rare cells retained by the microfluidic device. The one or more rare cells can divide and form daughter cells. The daughter cells can be collected and then divided into one or more sets of daughter cells. The daughter cells can be subjected to one or more therapeutic treatment. The one or more therapeutic treatments can be therapeutic treatments associated with the condition that was diagnosed, prognosed, or theranosed. For example, the condition can be ovarian cancer and the therapeutic treatment can include treatment of a first set of daughter cells with doxorubicin and treatment of a second set of daughter cells with gene therapy. The daughter cells can be analyzed before and after one or more therapeutic treatments using any analysis methods described herein. The analysis methods can include enumeration of the daughter cells. Non-proliferation or increased reduction in daughter cell numbers can indicate a preference for one treatment over another. A theranosis can be made based on the results of the analysis of the daughter cells before and after one or more therapeutic treatments.

### Business Methods and Kits

The invention also contemplates business methods for selling a service of diagnosis, theranosis, or prognosis of a condition in exchange for a fee. The diagnosis, theranosis, or prognosis can be based on one or more analysis methods described herein. The analysis methods can include enriching one or more rare cells (e.g., circulating epithelial cells) in a first sample (e.g., blood sample) obtained from a patient and performing a first analysis on the one or more rare cells (e.g., enumerating a subtype of the rare cell). The business may then provide results from the first analysis to a patient or care provider or insurance which would be combined with other information to make a prognosis or diagnosis. Optionally, the business may perform a second analysis on a second sample obtained from the patient. The second analysis can include detecting a serum marker or performing nucleic acid analysis on a biopsy. The business can then combine the results of the first and second analyses above and provide a single result to the patient, care provider, or insurance regarding the patient's diagnosis or prognosis.

Similarly, the business may provide information (in exchange for a fee) on potential therapies for the patient based on enriching rare cells using a microfluidic device having an array of obstacles, culturing the rare cells on the microfluidic device, subjecting the rare cells enriched on the device to one or more therapeutic treatments, and determining whether or not such treatments would be appropriate for the patients based on analysis of the cells treated.

The business also may sell kits that can be used to diagnose, theranose, or prognose a condition in a patient. The kit can include a microfluidic device comprising an array of obstacles optionally covered with one or more binding moieties; and one or more reagents for performing nucleic acid analysis (e.g., on biopsies), detecting a serum marker (e.g., any of the ones mentioned herein), and/or culturing cells. The kit can also comprise instructions for use and a container.

### Example 1

A Patient is Evaluated for the Presence or Absence of Prostate Cancer by Analyzing for a Serum Marker in a First Sample Taken from the Patient and by Enumerating the Number of Rare Cells in a Second Sample Taken from the Patient.

A blood sample is obtained from the patient and split into a first sample and a second sample. The first sample is analyzed for prostate specific antigen using a diagnostic kit for detecting levels of prostate specific antigen in a blood sample. A known quantity of blood is mixed with a reagent from the diagnostic kit that binds to prostate specific antigen forming a reaction mixture. The reaction mixture is applied to a test strip. The test strip is washed and a level of prostate specific antigen in the blood sample is reported by an indicator. The level of prostate specific antigen in the blood sample is recorded.

The second sample is applied to a microfluidic device comprising an array of obstacles and anti-EpCAM binding moieties. The array of obstacles can include multiple subarrays that are fluidly coupled to one another in series. The subarrays are arranged such that the average gap length between obstacles in a subarray decreases between each subarray and the next subarray downstream to it.

As sample flows through the microfluidic device, one or more rare cells are retained by the microfluidic device due to size and/or affinity. The number of rare cells retained by the microfluidic device is enumerated and recorded.

The presence or absence of prostate cancer is determined based on the level of prostate specific antigen in the blood sample and the number of rare cells retained by the microfluidic device.

### Example 2

A Patient is Evaluated for the Presence or Absence of Breast Cancer by Analyzing for a Nucleic Acid in a First Sample Taken from the Patient and by Enumerating the Number of Rare Cells in a Second Sample Taken from the Patient.

A biopsy sample and a blood sample are obtained from the patient. The biopsy sample is analyzed for a gene encoding ERBB2 using a RT-PCR for detecting levels of the ERBB2 gene expression in the biopsy sample. The level of ERBB2 gene in the biopsy sample is recorded.

The blood sample is applied to a microfluidic device comprising an array of obstacles and covered with anti-EpCAM binding moieties. The array of obstacles includes multiple subarrays that are fluidly coupled to one another. The subarrays are staggered such that they form a restricted gap between adjacent subarrays. Each subarray can have the same or a different average gap between its obstacles.

As sample flows through the microfluidic device, one or more rare cells are retained by the microfluidic device due to size and/or affinity interactions. The number of rare cells retained by the microfluidic device is enumerated and recorded.

The presence or absence of breast cancer is determined based on the level of ERBB2 gene expression in the biopsy sample and the number of rare cells retained by the microfluidic device. In some instances, at least 5, 10, 50 or 100 different gene expressions are assayed in combination with the enumeration of rare cells.

### Example 3

A Patient is Evaluated for the Presence or Absence of Cancer by Enumerating the Number of Circulating Tumor Stem Cells in a Sample Taken from the Patient.

A blood sample is obtained from the patient and applied to a microfluidic device comprising an array of obstacles and anti-EpCAM binding moieties. The array of obstacles has a uniform pattern such that each successive row is offset from the previous row by ½ the period with the exception of a subset of obstacles that are unaligned from the above pattern such that they form a restricted gap (smaller than the average gap size).

As sample flows through the microfluidic device, one or more CTCs are retained by the microfluidic device due to size and/or affinity interactions. The CTCs are detected using a label comprising an antibody to cytokeratin and a first detectable label. The first detectable label is phycoerythrin. A subset of the CTCs, the circulating tumor stem cells, are detected using an antibody to CD44 and a second detectable label that is distinct from the first detectable label. The second detectable label can be FITC. The number of CTCs and circulating tumor stem cells retained by the microfluidic device is enumerated and recorded. The CTC's and/or circulating tumor stem cells may be further assayed using various nucleic acid techniques such as qPCR, SNP, ultra-deep sequencing, mRNA analysis.

The presence or absence of cancer is determined based on the number of CTCs and circulating tumor stem cells retained by the microfluidic device and optionally from the nucleic acid analysis.

### Example 4

Therapeutic Treatment for a Patient with Breast Cancer is Evaluated by Enriching CTCs in a Blood Sample Obtained from the Patient and Subjecting the CTCs to Two Therapeutic Treatments.

A blood sample of 7.5 or 50 mL is obtained from the patient and applied to a microfluidic device comprising an array of obstacles and anti-EpCAM binding moieties. The array of obstacles includes multiple subarrays that are fluidly coupled to one another. The subarrays are arranged such that the blood sample contacts the multiple subarrays sequentially. Each subarray has a decreasing average gap length between obstacles as compared to the previous subarray (the one upstream from it).

As sample flows through the microfluidic device, one or more CTCs are retained by the microfluidic device due to size and/or affinity. The number and optionally average size of CTCs retained by the microfluidic device is enumerated and recorded. These numbers may be used to prognose or stage the breast cancer.

The microfluidic device is flooded with EpCAM antigen and then the CTCs are cultured on the microfluidic device by introducing a culture medium to the microfluidic device. Daughter CTCs slough off the microfluidic device and into the culture medium.

After one week, the unattached CTCs are harvested and split into two sets of circulating tumor cells. The first set of CTCs are subjected to therapeutic treatment by Her-2 antibodies and the second set of CTCs are subjected to treatment by RNAi therapy. Response of the CTCs to therapeutic treatment is monitored.

Selection of therapeutic treatment for the patient is determined by the response of the CTCs to the two therapeutic treatments.

1-18. (canceled)

19. A method for diagnosing, theranosing, or prognosing a condition in a subject, comprising:
a) enriching one or more rare cells from a sample obtained from said subject using a microfluidic device;
b) performing a first analysis of one or more cell subtypes of said one or more rare cells; and
c) evaluating the result of said first analysis to make said diagnosis, theranosis, or prognosis.

20. The method of claim 19, further comprising labeling one or more rare cells using a first label and labeling one or more cell subtypes using a second label.

21. The method of claim 20, wherein the first label is distinct from the second label.

22. The method of claim 20, wherein the first label and the second label have a light absorption wavelength or a fluorescence emission wavelength that is separated by more than 5, 10, 25, 30, 40, or 50 nm.

23. The method of claim 19, wherein the first analysis comprises enumerating the one or more cell subtypes.

24. The method of claim 19, wherein the cell subtypes comprise circulating tumor cells, circulating tumor stem cells, circulating stem cells, or stem cells.

25. The method of claim 19, wherein the microfluidic device comprises an array of obstacles and/or one or more binding moieties.

26. The method of claim 25, wherein the one or more binding moieties comprise anti-EpCAM.

27. The method of claim 19, further comprising subjecting said enriched one or more rare cells to one or more therapeutic treatments after step b), performing a second analysis of one or more cell subtypes, and evaluating the results of said first and second analysis to make said diagnosis, theranosis, or prognosis.

28. The method of claim 19, wherein steps a)-c) are performed at a first time and a second time, and the results obtained from at the first time and the results obtained at the second time are evaluated to make said diagnosis, theranosis, or prognosis.

29. The method of claim 28, further comprising subjecting said patient to one or more therapeutic treatments between said first time and said second time.

30-37. (canceled)
